(19) United States
(12) Patent Application Publication
(10) Pub. No.: US 2014/0214018 A1
(43) Pub. Date: Jul. 31, 2014

(54) METHOD AND SYSTEM FOR TISSUE MODULATION

(71) Applicants: Boaz Behar, Ganei-Tikva (IL); Shmuel Ben-Ezra, Pardes-Hanna (IL)

(72) Inventors: Boaz Behar, Ganei-Tikva (IL); Shmuel Ben-Ezra, Pardes-Hanna (IL)

(73) Assignee: Perseus-BioMed Inc., Orangeburg, NY (US)

(21) Appl. No.: 14/342,393

(22) PCT Filed: Sep. 2, 2012
(86) PCT No.: PCT/IB2012/054524

§ 371 Date: Mar. 3, 2014

Related U.S. Application Data
(60) Provisional application No. 61/529,936, filed on Sep. 1, 2011.
(60) Provisional application No. 61/605,237, filed on Mar. 1, 2012.

(51) Int. Cl. A61B 18/08

(52) U.S. Cl. CPC A61B 18/082
USPC 606/28

(57) ABSTRACT

A method of modulating tissue of an internal organ in vivo is disclosed. The method comprises: fixating the tissue on a shaped device so as to shape the tissue generally according to a shape of the device; and focusing radiation on the fixated tissue using a radiation-emitting system so as to modulate the tissue, wherein the radiation-emitting system is non-local with respect to the shaped device.

### RELATED APPLICATIONS

This application claims priority from U.S. Provisional Patent Application No. 61/529,936 filed Sep. 1, 2011, and U.S. Provisional Patent Application No. 61/605,237 filed Mar. 1, 2012. Both documents are incorporated herein by reference in their entirety.

### FIELD AND BACKGROUND OF THE INVENTION

The present invention, in some embodiments thereof, relates to medical devices and techniques and, more particularly, but not exclusively, to a method and system useful for tissue modulation by delivering energy to the tissue or removing energy from the tissue.

There are many medical situations where tissue modulation or damage has been demonstrated to be clinically beneficial. Device-based approaches for tissue modulation include treatment of tissue by energy treatments, such as high intensity focused ultrasound (HIFU), cryotherapy, and treatment of tissue by electromagnetic radiation of various spectra, including X-Ray, microwave, radiofrequency (RF) and the like.

Tissue modulation treatments have heretofore been employed for many types of conditions and pathologies. Known in the art are thermal treatments of the prostate, and reduction of the nerve activity (also known as denervation) in cases of hyperactivity of the sympathetic nervous system.

For example, it has been demonstrated (Renal Sympathetic-Nerve Ablation for Uncontrolled Hypertension, New England Journal of Medicine, 2009; 361:932-934, Aug. 27, 2009) that denervation by energy treatment of renal artery nerves, can reduce blood pressure in uncontrolled hypertension patients. It is believed that such a treatment has additional clinical benefits, such as increasing insulin uptake.

U.S. Published Application No. 20070129760 the contents of which are hereby incorporated by reference, describes a technique in which a neural denervation element is positioned within a blood vessel of a patient, and activated to denervate the tissue that is innervated by neural matter located within or in proximity to the blood vessel. The neural denervation element is configured to deliver thermal energy, high intensity focused ultrasound (HIFU) or neuromodulatory agent to the neural tissue.

U.S. Pat. No. 5,492,126 the contents of which are hereby incorporated by reference, describes combining ultrasound image guidance with HIFU treatment for achieving improved accuracy in the treatment of tissue.

U.S. Pat. No. 6,576,875 the contents of which are hereby incorporated by reference, describes a method of ultrasound spectral analysis imaging of the treatment beam correlated with the tissue imaging to allow imaging of both tissues and beam.

U.S. Pat. No. 6,128,522 the contents of which are hereby incorporated by reference, describes an MRI guidance method for heat treatment methods such as HIFU.

Additional background art includes U.S. Pat. No. 7,630,750.

### SUMMARY OF THE INVENTION

According to an aspect of some embodiments of the present invention there is provided a method of modulating tissue of an internal organ in vivo. The method comprises: fixating the tissue on a shaped device so as to shape the tissue generally according to a shape of the device; and focusing radiation on the fixated tissue using a radiation-emitting system so as to modulate the tissue, wherein the radiation-emitting system is non-local with respect to the shaped device. According to some embodiments of the invention the method wherein the radiation-emitting system is a non-invasive radiation-emitting system.

According to some embodiments of the invention the method wherein the radiation-emitting system is a minimally-invasive radiation-emitting system introduced into an organ other than the organ hosting the shaped device.

According to some embodiments of the invention the radiation comprises high intensity focused ultrasound (HIFU).

According to some embodiments of the invention the radiation selected from the group consisting of X-ray and microwave.

According to some embodiments of the invention the method further comprises scanning the focused radiation along a predetermined path corresponding to the shape of the device so as to form a modulation pattern on the tissue.

According to some embodiments of the invention the scanning comprises moving the radiation-emitting system.

According to some embodiments of the invention the scanning is effected by a phased array radiation-emitting system.

According to some embodiments of the invention the method further comprises receiving signals indicative of a relative position of the radiation-emitting system with respect to the shaped device, wherein the scanning is responsively to the relative position.

According to some embodiments of the invention the method further comprises sensing the radiation at or in proximity to the shaped device, and correcting the path responsively to the sensing.

According to some embodiments of the invention the shaped device comprises a sensor operable to detect and report energy transmitted by the radiation-emitting system.

According to some embodiments of the invention the shaped device comprises a reflector operable to reflect energy transmitted by the radiation-emitting system.

According to some embodiments of the invention the shaped device comprises a portion sized and shaped to deploy as a helix biased against an inner wall of a blood vessel.

According to some embodiments of the invention the shaped device comprises an expandable portion sized and shaped to bring elements of the expandable portion into contact with an inner wall of a blood vessel when the expandable portion is expanded within the blood vessel.

According to some embodiments of the invention the expandable portion is constrained to a narrow configuration while being advanced through a blood vessel, and is opened into an expanded configuration when positioned at a treatment site.

According to some embodiments of the invention the expandable portion is constrainable to a narrow configuration by a containing sheath, and is openable to an expanded configuration by one of extending the expandable portion beyond a distal portion of the sheath, and retracting a distal portion of the sheath from around the expandable portion.

According to some embodiments of the invention the expandable portion is made to assume the expanded configuration by one of mechanical, thermal and electrical activation.

According to some embodiments of the invention the sensing is performed selectively at a plurality of discrete locations.

According to some embodiments of the invention the sensing comprises reflecting the radiation outwardly and collecting the reflected radiation outside the body.

According to some embodiments of the invention the method comprises modulating the reflected radiation so as to encode spatial information therein.

According to some embodiments of the invention, modulating of the reflected radiation comprises periodically modifying reflectivity of a reflector with respect to energy arriving from a particular direction.

According to some embodiments of the invention, the method further comprises modifying reflectivity of a plurality of reflectors with differing periodicity.

According to some embodiments of the invention the method further comprises operating a data processor to execute an image analysis procedure so as to identify focal regions corresponding to the focused radiation.

According to some embodiments of the invention the imaging is performed intracorporeally.

According to some embodiments of the invention the method further comprises calibrating the radiation responsively to the sensing.

According to some embodiments of the invention the method comprises receiving prerecorded calibration data having a plurality of entries, each entry comprises a set of radiation parameters associated with a three-dimensional coordinate, and searching the data for three-dimensional coordinate corresponding to a sensing location to extract a respective set of radiation parameters, wherein the calibrating is also based on the respective set of radiation parameters.

According to some embodiment of the invention the method comprises receiving prerecorded calibration data having a plurality of entries, each entry comprises a set of radiation parameters associated with a three-dimensional coordinate, and searching the data for radiation parameters received by sensor, for its corresponding three-dimensional coordinate.

According to some embodiments of the invention the method further comprises, prior to the modulation of the tissue, operating the radiation-emitting system to emit non-damaging radiation, wherein the correction of the path is performed during the emission of the non-damaging radiation.

According to some embodiments of the invention the method comprises, prior to the modulation of the tissue, operating the radiation-emitting system to emit non-damaging radiation, wherein the calibration is performed during the emission of the non-damaging radiation.

According to some embodiments of the invention the method comprises repeating the emission of the non-damaging radiation and the modulation intermittently.

According to some embodiments of the invention at least one of: a rate and a duty cycle of the intermittent repetition is selected to match one of a characteristic breathing cycle of a subject having the organ, a heartbeat, movement of a digestive organ, a patient movement, or a combination of any these.

According to some embodiments of the invention the predetermined path forms a non-closed loop spanning, optionally in a helical pattern, about a longitudinal axis, and optionally spanning between 90° and 540°.

According to some embodiments of the invention the predetermined path generally forms a helix.

According to some embodiments of the invention the tissue is a nerve and the modulation comprises denervation. According to some embodiments of the invention the nerve is a part of an autonomic nervous system. According to some embodiments of the invention the nerve is selected from the group consisting of a nerve leading to a kidney, a sympathetic nerve connected to a kidney, an afferent nerve connected to a kidney, an efferent nerve connected to a kidney, a renal nerve, a renal sympathetic nerve at a renal pedicle, a nerve trunk adjacent to a vertebra, a ganglion adjacent to a vertebra, a dorsal root nerve, an adrenal gland, a motor nerve, a nerve next to a kidney, a nerve behind an eye, a celiac plexus, a nerve within a vertebral column, a nerve around a vertebral column, nerve extending to a facet joint and a celiac ganglion. According to some embodiments of the invention the nerve is a renal artery nerve.

According to some embodiments of the invention the tissue is a prostatic tissue in the prostate.

According to some embodiments of the invention the organ is selected from the group consisting of prostate, liver, kidney, pancreas and heart.

According to an aspect of some embodiments of the present invention there is provided a catheter system. The system comprises: a shaped device adapted for being introduced into a living body and being configured for fixating a tissue thereon so as to shape the tissue generally according to a shape of the device; and at least one passive ultrasound sensor mounted on the device and configured for sensing at least one of: a position of the shaped device within a living body, and radiation emitted by an ultrasound radiation-emitting system which is non-local with respect to the device and which is optionally external to the body. Additionally or alternatively, the system comprises a device suitable for being positioned in tissue and expandable to a generally known shape of the tissue.

According to some embodiments of the invention there is provided a shaped device adapted for being introduced into a living body and being configured for fixating a tissue thereon so as to shape the tissue generally according to a shape of the device; and at least one passive ultrasound sensor mounted on the device and configured for sensing at least one of: a position of the shaped device within a living body, and radiation emitted by an ultrasound radiation-emitting system non-local with respect to the device and optionally external to the body.

According to some embodiments of the invention the system wherein the radiation comprises high intensity focused ultrasound (HIFU).

According to an aspect of some embodiments of the present invention there is provided a system for modulating tissue of an internal organ in vivo. The system comprises: a shaped device adapted for being introduced into a living body and being configured for fixating a tissue thereon so as to shape the tissue generally according to a shape of the device; a radiation-emitting system configured for emitting radiation from a location external to the body and focusing the radiation on the fixated tissue; a scanning system operative to scan the radiation over the fixated tissue; and a controller, configured for controlling the radiation-emitting system and the scanning system such that the scan is along a predetermined path corresponding to the shape of the device so as to from a modulation pattern on the tissue.

According to some embodiments of the invention the system comprises at least one sensor mounted on the shaped device and configured for sensing at least one of: a position of the shaped device within a living body, and radiation emitted by the radiation-emitting system, wherein the controller is configured for receiving signals from the at least one sensor and for controlling the radiation-emitting system and the scanning system, responsively to the signals.

According to some embodiments of the invention the at least one sensor comprises a plurality of sensors arranged at a plurality of discrete locations over the shaped device.

According to some embodiments of the invention the system comprises: at least one reflector mounted on the shaped device and configured for reflecting radiation emitted by the radiation-emitting system; and at least one radiation sensor configured for sensing the reflected radiation at one or more sensing locations external to the shaped device; wherein the controller is configured for receiving signals from the at least one radiation sensor and for controlling the radiation-emitting system and the scanning system, responsively to the signals.

According to some embodiments of the invention the at least one radiation sensor comprises a sensors adapted to be located outside the body.

According to some embodiments of the invention the at least one radiation sensor comprises a sensors adapted to be located inside the body but external to the device.

According to some embodiments of the invention the reflectors are configured for modulating the reflected radiation to encode spatial information therein, wherein the controller is configured for extracting the spatial information from the signals, and for controlling the radiation-emitting system and the scanning system responsively to the extracted spatial information.

According to some embodiments of the invention the controller is configured for calibrating the radiation responsively to the sensing.

According to some embodiments of the invention the controller is configured to access prerecorded calibration data having a plurality of entries, each entry comprises a set of radiation parameters associated with a three-dimensional coordinate, and to search the data for three-dimensional coordinate corresponding to a sensing location so as to extract a respective set of radiation parameters, wherein the calibrating is also based on the respective set of radiation parameters.

According to some embodiments of the invention the controller is configured to access prerecorded calibration data having a plurality of entries, each entry comprises a set of radiation parameters associated with a three-dimensional coordinate, and to search the data for radiation sensing parameters to determine its corresponding three-dimensional coordinates.

According to some embodiments of the invention the system comprises: an intracorporeal imaging system configured for imaging the fixated tissue and regions in proximity thereto, wherein the controller is configured for analyzing imagery data received from the intracorporeal imaging system, and identifying focal regions corresponding to the focused radiation.

According to some embodiments of the invention the shaped device comprises a portion sized and shaped to deploy as a helix biased against am inner wall of a blood vessel.

According to some embodiments of the invention the shaped device comprises an expandable portion sized and shaped to bring elements of the expandable portion into contact with an inner wall of a blood vessel when the expandable portion is expanded within the blood vessel.

According to some embodiments of the invention the system comprises a mechanism which constrains the expandable portion to a narrow configuration and which opens the expandable portion into an expanded configuration, under control of a user.

According to some embodiments of the invention the mechanism is a constraining sheath which constrains the expandable portion only when the expandable portion is contained within a distal portion of the sheath.

According to some embodiments of the invention the mechanism causes the expandable portion to expand in response to one of mechanical, thermal and electrical activation.

According to an aspect of some embodiments of the present invention there is provided a system for modulating tissue of an internal organ in vivo. The system comprises: a shaped device adapted for being introduced into a living body; a radiation-emitting system configured for emitting radiation from a location external to the body and focusing the radiation on the shaped device; at least one sensor mounted on the device, and being configured for sensing the radiation; and a data processor, configured for analyzing signals received from the at least one sensor and calculate at least one of: a relative location and a distance of a focal region of the radiation.

According to some embodiments of the invention the system wherein the radiation-emitting system configured for scanning the tissue and wherein the data processor is also configured for calculating a scanning path of the focal region.

According to some embodiments of the invention the data processor is configured for receiving a geometric relationship between the at least one sensor and a shape of the tissue, and to calculate the relative location and/or the distance based, at least in part, on the geometric relationship.

According to some embodiments of the invention the data processor is configured for calculating geometric relationship between the at least one sensor and a shape of the tissue, and to calculate the relative location and/or the distance based, at least in part, on the geometric relationship.

According to some embodiments of the invention a system includes multiple sensors mounted on multiple devices positioned in vivo, and a processor configured for calculating a treatment path of a beam to fit a geometry according to which the beam will not harm tissue located at position known relative to these multiple sensors.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for modulating tissue of an internal organ in vivo, according to some exemplary embodiments of the present invention;

FIG. 2 is a schematic illustration of renal nerves;

FIG. 3 is a flowchart diagram describing the method of the present embodiments in greater detail;

FIG. 4A is a schematic illustration of a catheter system, according to some embodiments of the present invention;

FIG. 4B is a schematic illustration of a shaped device according to some embodiments of the present invention; and

FIG. 5 is a schematic illustration of a system for modulating tissue of an internal organ in vivo, according to some embodiments of the present invention.

### DESCRIPTION OF SPECIFIC EMBODIMENTS OF THE INVENTION

The present invention, in some embodiments thereof, relates to medical devices and techniques and, more particularly, but not exclusively, to a method and system useful for tissue modulation by delivering energy to the tissue or removing energy from the tissue.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It is recognized that the treatment of tissues by remote, focused, energy methods such as X-Ray radiation, microwave radiation, ultrasound radiation and alike, generally requires that the energy beam be directed and/or focused onto the tissue. It is desired that the direction of energy be inline with the position and shape of the organ or tissue to be treated, so as to effectively treat the tissue, preferably with minimal or no damage to neighboring tissues.

When using, for example, ultrasound such as, but not limited to, high intensity focused ultrasound (HIFU), the direction of the ultrasonic beam entering the body may not assure hitting the target tissue, since the ultrasonic energy passes through multiple tissue segments, experience multiple different ultrasonic speed, and therefore deflections in directions which are a priori unknown. Another cause for misalignment between the energy beam and the target tissue relates to tissue motion which results in continuous variation of the spatial relations between the tissue and the radiation system. Beam misalignment and unpredicted phase errors can cause focus dispersion and reduced treatment efficacy, because the more concentrated the energy is, the better the chances for successful denervation, but in dispersed beams, the available energy is spread over a larger area and weaken the therapeutic effect.

Heretofore, image guidance of HIFU has been utilized using MRI and ultrasound, typically tuning the direction and path of the energy beam at low, non harmful energy levels, and turning the power up for treatment once in position.

MRI can be used as a technique for measuring intrabody temperature noninvasively. When using MRI to guide HIFU for example, the HIFU beam can be tuned to a low, non harmful energy level, and the MRI image can image the tissue shape and position, as well as the beam focus via the temperature imaging capability of MRI.

It was found by the present inventors, that conventional MRI guided heat treatments have several drawbacks. For example, for use in real time, all of the equipment used within the procedure needs to be non ferromagnetic, substantially increasing the complexity and price of auxiliary equipment. Additionally, MRI has a relatively slow shutter time, causing blurriness over moving organs such as arteries and veins in the vicinity of the lungs.

Traditional diagnostic ultrasound is known to be suitable for imaging the target tissue location and shape. The present inventor recognized that this modality is not capable of imaging the HIFU beam because this beam does not create an echo which is different from the tissue it passes through. Known techniques for imaging the HIFU beam include: imaging of mechanical artifacts of the beam, analysis of speckles, and spectral analysis.

It was found by the present inventors, that conventional ultrasound guided heat treatments have several drawbacks. For example, when the guidance is based on mechanical artifacts, it is necessary to induce such artifacts, which are oftentimes not desirable, in particular when the beam interacts with tissues other that the target tissue. When the guidance is based on imaging of speckles or spectral analysis, the spatial resolution of the location of the beam is confined to 3-10 wavelengths. This results in a bad resolution, or requires very high frequencies which reduce the effective imaging range.

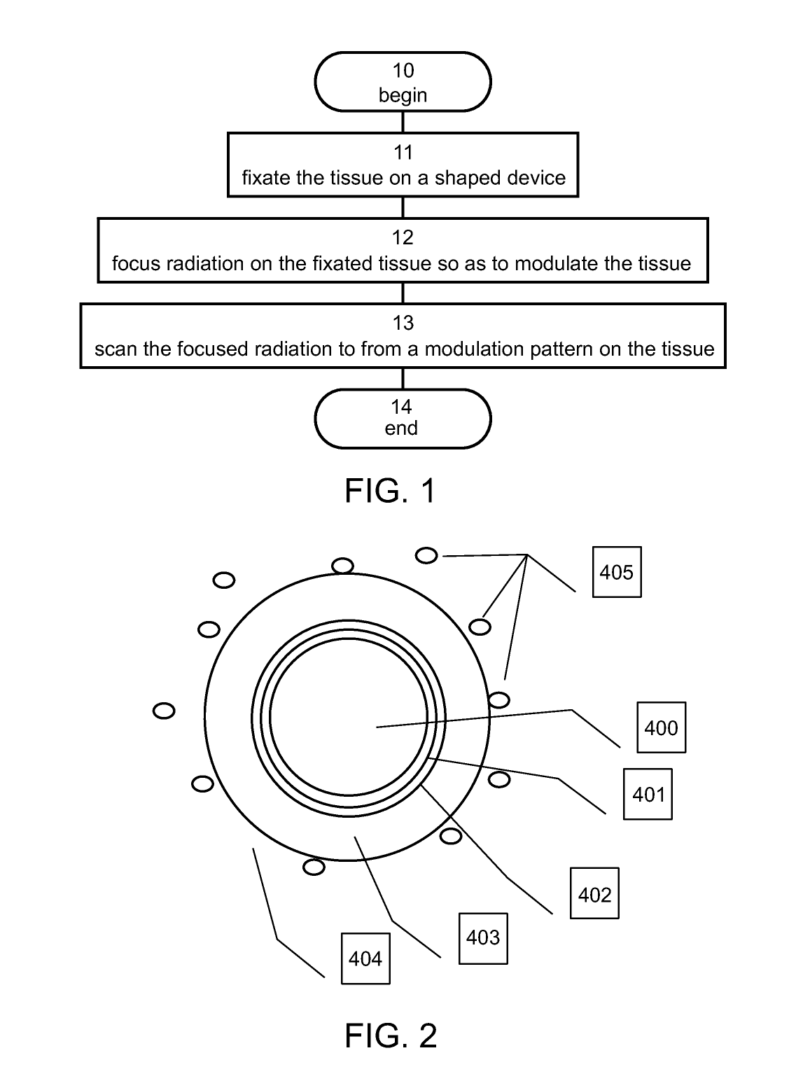

Referring now to the drawings, FIG. 1 is a flowchart diagram of a method suitable for modulating tissue of an internal organ in vivo, according to some embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and continues to 11 at which a target tissue is fixated on a shaped device so as to shape the tissue generally according to a shape of the device.

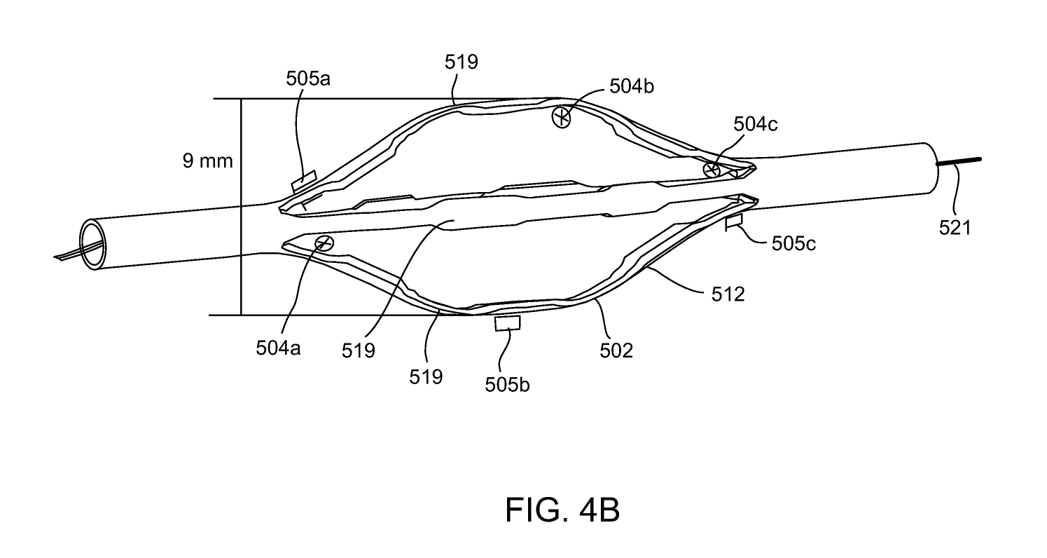

The term “fixated” is used herein to specify a condition in which a tissue is immobilized with respect to a shaped device. For example, a device such as that shown in FIG. 4B may fixate a tissue by expanding within that tissue (e.g. an artery) until that tissue is pressured or somewhat stretched by the device, and is thereby immobilized. By this process the tissue may also be constrained into a predetermined shape corresponding to the shape of the shaped device. Any other method of attaching a tissue to a device is also be considered as ‘fixating’ that tissue with respect to the device. However, it is noted here that altering the geometry of the tissue being ‘fixated’ is not a requirement of the invention; any arrangement which immobilizes one with respect to the other is to be understood as included in the meaning of “fixated” as that term is used in the this disclosure and in the accompanying claims. For example, a device which expands within a lumen so as to assume a known geometrical shape of that lumen would be considered “fixated”, as that term is used herein, if lumen and expandable device were thereby temporarily immobilized one with respect to the other, even if both are moving in absolute space, e.g. as a result of a heartbeat or respiration. In some embodiments, a shaped device is an expandable device operable to expand to at least approximately match a shape and size of at least a part of said tissue.

Note also that the terms the terms “energy projector” and “energy transmitter” and “energy emitter”, as used herein, are considered to have the same meaning and are used interchangably.

The target tissue may be any type of internal tissue. Representative examples include, without limitation, a nerve tissue, particularly a nerve which is a part of an autonomic nervous system, a prostate tissue, a liver tissue, a kidney tissue, a pancreas tissue and a heart tissue.

Nerve tissues suitable for some embodiments of the present invention include, without limitation, a nerve leading to a kidney, an efferent nerve leading from the kidney, a renal nerve, a sympathetic nerve connected to a kidney, an afferent nerve connected to a kidney, a renal sympathetic nerve at a renal pedicle, a nerve trunk adjacent to a vertebra, a ganglion adjacent to a vertebra, a dorsal root nerve, an adrenal gland, a motor nerve, a nerve next to a kidney, a nerve behind an eye, a celiac plexus, a nerve within a vertebral column, a nerve around a vertebral column, a nerve extending to a facet joint, a celiac ganglion, a cardiac nerve, a portion of a brain. Some embodiments of the invention also treat cancerous tissue in or near a lumen, such as for example a blood vessel, in which a catheter may be placed.

In some embodiments of the present invention the tissue is a renal artery nerve, and in some embodiments of the present invention is the tissue is a renal vein nerve.

The shaped device is preferably adapted for being introduced into the body, for example, in an endoscopic, laparoscopic or intravascular manner. Typically, the shaped device is provided as, or being mounted on, a catheter system, which may be endoscopic, laparoscopic or intravascular. The shaped device may have any shape that the tissue can assume. For example, the device may have a generally cylindrical shape (e.g., a cylindroid, a circular cylinder etc.). Other shapes include, without limitation, a spiral, a helix, a disk, an oval, a cuboid, a prism, a sphere, a hemisphere, a portion of a sphere, a spheroid, a portion of a spheroid, a prolate spheroid, an oblate spheroid, an ellipsoid, a portion of ellipsoid, a hyperboloid, a portion of a hyperboloid, a paraboloid, a portion of a paraboloid, a cylindrical shell, a portion of a cylindrical shell, a polyhedron shell, a portion of a polyhedron shell, and any combination between two or more of these shapes.

The tissue may be fixated on the device by any technique known in the art. For example, the shaped device may be made expandable, wherein its expanded shape is preplanned. The device may assume its expanded shape in response to external activation (e.g., mechanical, thermal and/or electrical activation).

A representative example is an expandable mesh (e.g., a stent or the like) that upon expansion shapes the blood vessel co-axially to a catheter. (An example of such a structure is discussed hereinbelow with respect to FIG. 4B.) Also contemplated are embodiments in which the size and/or shape of the device is adapted to the target tissue of the specific subject. In these embodiments, the method first receives data pertaining to the size and/or shape of the target tissue in its relaxed state, and determines the shape of the device based on the received data. For example, when the target tissue is a blood vessel, the data may include the diameter of the blood vessel, and the shape of the device may be selected to be a cylinder or cylindroid having a diameter which is slightly larger that the diameter of the blood vessel in its relaxed state. Data pertaining to the shape and/or size of the tissue in its relaxed state may be acquired, for example, by imaging.

In some embodiments of the present invention the shaped device has an expandable shape and the method measures the parameters of the shape (e.g., radius) once expanded. Such measurement may be performed by imaging or by a measuring device mounted on the shaped device and configured to communicate with an external system such as a controller of a radiation-emitting system or the like. In other embodiments, the shape is of a pre-defined nature such as a cylindrical shape, and some of its geometrical parameters, such as a diameter of the cylinder, are estimated by measurements of sensors placed on the cylinder (for example 4 non-collinear sensors positions).

The present inventors also contemplate a catheter made to deploy in a helical shape inside the blood vessel, such that its helix is biased against the inner wall of the blood vessel, as shown in FIG. 4A. The helical shape may include a fraction of a helix turn (e.g., half a turn) or it may include one or more helix turns (e.g., at least two turns). For blood vessels with smaller diameter, the overall length of the helical shape is optionally longer than for blood vessels with larger diameter. This may be achieved by providing the helical shape with a larger pitch and/or larger number of rounds. When the target tissue is the prostate or part thereof, the shaped device may be aligned to a typical urethra geometry, or to a urethra geometry that is specific to the subject.

The fixation may be applied to part of the tissue, leaving other parts of the tissue not fixated. For example, in the case of a helical shaped fixation, the internal blood vessel wall touching the helix may be fixated to the helical shape, while the opposite side to the helix of the vessel may be non-fixated.

The method continues to 12 at which radiation is focused on the fixated tissue so as to modulate the tissue.

The term “modulating” as used herein, refers to a change in the biological function or activity of the tissue, including, without limitation, proliferation, secretion, adhesion, apoptosis, cell-to-cell signaling, and the like. In some embodiments, the modulation at least partially damages the tissue, so as to abrogate, inhibit (partially or completely), slow and/or reverse the progression of a condition. In some embodiments of the present invention the modulation is made to alter a measurable condition. In some embodiments of the present invention the modulation comprises denervation. In some embodiments of the present invention the modulation includes modulating prostate tissue at a pre-planned distance from an urethra, e.g., for treating BPH or the like. In some embodiments of the present invention the modulation includes treatment of atrial fibrillation of the heart by modulating the pulmonary vein entrance to the heart. Other modulations are not excluded from the scope of the present invention.

As used herein, “denervation” refers to the modulation of a nerve so as to induce partial ablation, complete ablation or paralysis of that nerve.

The radiation focused at 12 is optionally and preferably performed using a radiation-emitting system which is non-local with respect to the shaped device. For example, the radiation-emitting system may be a non-invasive radiation-emitting system which is located outside the body. Also contemplated, are embodiments in which the radiation-emitting system is a minimally-invasive radiation-emitting system introduced into an organ other than the organ hosting the shaped device. For example, the shaped device may be introduced to one blood vessel and the radiation-emitting system may be introduced into another blood vessel. Another example is a configuration in which the shaped device is introduced to a blood vessel and the radiation-emitting system is introduced into the esophagus. An additional example is a configuration in which the shaped device is introduced into the urethra and the radiation-emitting system is introduced into the rectum.

Representative examples of radiation types suitable for the present embodiments include, without limitation, HIFU, X-ray, microwave and radiofrequency (RF). In some embodiments of the present invention the radiation is HIFU.

In some embodiments of the present invention the method continues to 13 at which the focused radiation is scanned along a predetermined path corresponding to the shape of the device so as to from a modulation pattern on the tissue. The scanning may be done by moving the radiation-emitting system and/or by diverting the radiation beam using an arrangement of redirecting elements, such as, but not limited to, mirrors, prisms, diffractive elements and the like. Also contemplated is the use of phased array elements. In these embodiments, the scanning is effected by altering the relative phase of the phased array elements.

In various exemplary embodiments of the invention the scanning is performed automatically, e.g., using a controller, based on the predetermined path.

The predetermined path may have any shape. In some embodiments of the present invention the predetermined path forms a non closed loop spanning over 360 degrees about a longitudinal axis. A repetitive example is a helix. A helix is particularly useful when the shape of the fixated tissue is elongated and it is desired to modulate the tissue from all sides. The parameters of the path are optionally and preferably based on the shape of the device to which the tissue is fixated. The number of these parameters is preferably sufficient to define the shape and size of the device. For example, when the shape is a cylinder, the parameters may include diameter and length; when the shape is helical, the parameters may include diameter, pitch and number of turns, etc.

When the method measures the geometrical parameters of the shaped device, once expanded, the size of the path is preferably selected also based on this measurement.

In some embodiments of the present invention, the geometry of the path is selected based on one or more parameters, other than the shape of the tissue. Representative examples of such parameters include, without limitation, the BMI of the patient, the required blood pressure decrease, the age of the patient, the gender of the patient, the weight of the patient, the blood pressure, the insulin absorption level and/or any other parameter of the patient or target tissue. For example, the diameter of the modulation path may be selected based on the BMI, wherein for patients with high BMI the diameter is higher than for patients with low BMI. As a representative example, for a patient with BMI above 30, the treatment path may be at a distance about 2 mm from the inner wall of the blood vessel, for a patient with BMI less than 20, the treatment path may be at a distance about 0.5 mm from the inner wall of the blood vessel, and for other patients, the treatment path may be at a distance above 2 mm and less than 0.5 mm from the inner wall of the blood vessel.

The path may be a continuous path (e.g., a line in three-dimensions along which the focal region of the radiation moves), or it may be a discrete path (e.g., a set of points on the target tissue which are sequentially visited by the focal region). The path may also be selected such that the focal region moves over a surface or a volume. The treatment of multiple points along the path may be sequential, simultaneous, or any combination thereof.

In some embodiments, the path includes multiple treatment points in closed proximity thereamongst such that the treatment at these multiple treatment points is executed at a single positioning of the shaped device. For example, when the treatment is via RF, multiple treatment RF electrodes are mounted in closed proximity to each other on the shaped device.

The path may enclose the entire organ or part thereof. For example, when the organ is a blood vessel, the path may enclose the entire periphery of the blood vessel, in which case the azimuthal angle φ, describing the path, satisfies 0≦φ≦360°, or only a portion of the periphery in which case φ satisfies φ₁≦φ≦φ₂, where φ₂−φ₁<360°, e.g., φ₂−φ₁=60° or φ₂−φ₁=90° or φ₂−φ₁=120° or φ₂−φ₁=150° or φ₂−φ₁=180° or φ₂−φ₁=210° or φ₂−φ₁=240° or φ₂−φ1=270°.

In some embodiments the modulation path has a non-closed shape, e.g., a shape other that a closed annular shape, and in some embodiments the modulation along the path is performed intermittently. These embodiments are particularly useful when the organ is a blood vessel and it is desired to maintain its physical strength. For example, when the path has a helical shape, two or more treatment cycles are implemented. In some of these cycles, the tissue is modulated along arc sections of the helix, wherein the arcs are characterized by an azimuthal angle φ which is sufficiently less than 360° (e.g., 10°, 20°, 30°, 40°, 50°, 60°, or any other angle) thereby leaving the complementary arc sections untreated. In other cycles, the modulation may be continued along other arc sections. The arc sections are preferably selected so as not to form a continuous closed path of treatment points along the tissue, thereby preventing the formation of a contour of mechanical weakness along the blood vessel.

In some embodiments of the present invention the treatment path is visualized on a display device thereby allowing the physician to follow the path manually. When the scanning is performed automatically, the visualized path may be used by the physician for control and adjustment purposes.

The method ends at 14.

Before providing a further detailed description of the method according to some embodiments of the present invention, attention will be given to the advantages and potential applications offered thereby.

The method of the present embodiments is useful particularly, but not exclusively, for the denervation of nerves leading to a kidney, such as, but not limited to, a renal nerve.

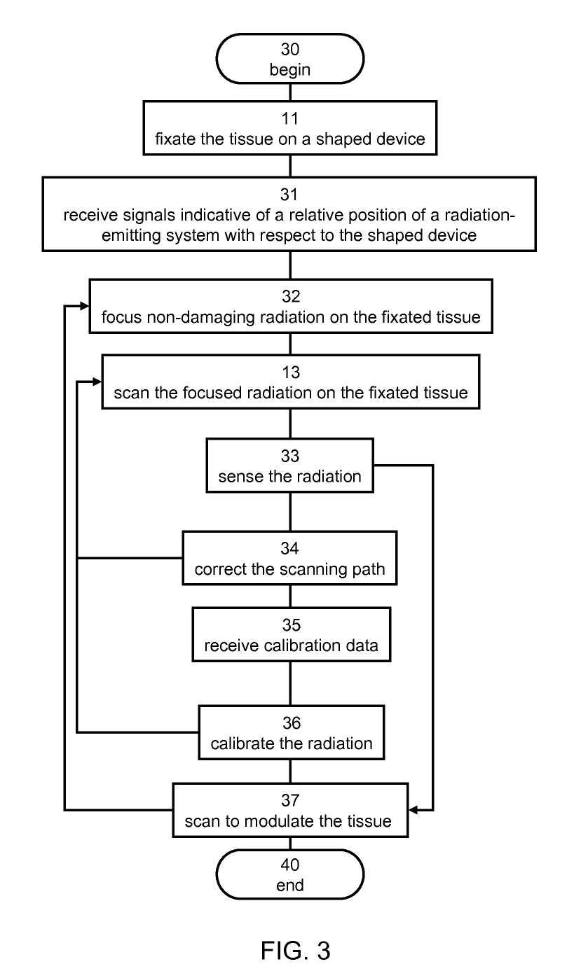

The renal nerves are aligned at the periphery of the renal arteries and renal veins. A schematic illustration of renal nerves is shown in FIG. 2. Shown in FIG. 2 is a cross section of a blood vessel (artery or vein), having an inner lumen 400 which is occupied by blood (not shown), an inner vessel wall 401 known as the tunica intima, an elastin layer 402, a bulk wall layer 403 known as tunica media, and an outer layer 404 known as tunica adventita. Elastin layer 402 is between inner wall 401 and bulk wall layer 403, and outer layer 404 surrounds bulk wall layer 403. Outer layer 404 is inervated by the sympathetic nerves 405, which surround the blood vessel generally from all sides at a distance of from about 0.5 mm to about 3 mm away from inner wall 401.

Elastine layer 402 grants the blood vessel some of its mechanical flexible capacity. It is generally preferred to modulate the renal nerves, typically a major part thereof, without or with minimal damage to the layers forming the wall of the blood vessel, particularly inner wall 401 and elastin layer 402, so as to prevent, minimize or at least reduce risk of hemorrhage, tearing, or breaking of the blood vessel.

Conventional techniques for the ablation of renal artery nerves are based on intravascular catheter RF modulation. In this procedure, a catheter is inserted to the required spot of ablation in the artery, and RF energy is induced to the tissue from within the artery using electrodes (see, e.g., U.S. Pat. No. 7,756,583, the contents of which are hereby incorporated by reference). The present inventors found that RF ablation for the treatment of renal nerve has several drawbacks. An intravascular catheter based RF signal needs to substantially heat at the point of contact, in order to affect the target nerve, since it is not capable of generating heat other than at touch-point. To reach physiological ablation of a nerve a temperature of about 60° is typically required for a time period of a few seconds to minutes. For such amount of heat to reach the nerve, the temperature at the point of contact between the catheter and the inner wall 401 should be about 70°. This, however, results in damage to the inner layers of the blood vessel, particularly the inner wall 401 and elastin 402. Additionally, due to substantial heat losses, such an approach in limited only to nerves being very close (about 1 mm or less) to outer wall 404, while it is recognized by the present inventors that renal nerves, for example, are located at deeper depths from the arterial wall.

Another conventional technique includes use of intravascular HIFU catheters (see, e.g., U.S. Published Application No. 20110112400, the contents of which are hereby incorporated by reference). In this technique, a HIFU transducer is built at the distal end of a catheter, so as to transmit energy to the renal nerves. The present inventors found that intravascular HIFU for the treatment of renal nerve has several drawbacks. HIFU transmitters can create extensive damage when the energy flux on the contact area of the transducer is too high. Common safety standards limit such a flux not to exceed 3 w/cm2. Therefore, HIFU transducers are limited in the total transmitted energy by the size of the contact area between the transducer and the contact tissue. The renal artery at the area of treatment may be quite narrow, typically no more than 6 to 7 mm in diameter [“Original Research: MDCT Angiography of the Renal Arteries in Patients with Atherosclerotic Renal Artery Stenosis: Implications for Renal Artery Stenting with Distal Protection”, American Journal of Roentgenology, June 2007, Vol. 188:6, pp. 1652-1658], and this substantially limits the amount of energy that an internal transducer can transmit. The size of the blood vessel hence imposes a minimal contact area between the transducer and the inner blood vessel wall (no more than 1 to 2 cm2) and therefore substantially limits the outwardly depth of treatment due to wave attenuation.

The attenuation of ultrasound waves in tissue may be expressed as exp(α₀ f^{1.2} x), where α0 is a coefficient, f is the frequency of the wave, and x is the propagation distance. In order to be able to control direction and focus of ultrasonic energy, the components of the internal ultrasound system are typically a few wavelengths in size. According to calculations performed by the present inventors, the required frequency for an internal HIFU system is generally high. For example, for a 7 mm diameter of blood vessel required frequency is typically about 5 MHz or more. High frequency, however, imposes a very short effective distance from the ultrasound source, in particular with a low power transducer. Thus, according to the above calculations the use of intravascular HIFU is generally ineffective.

Another drawback of conventional intravascular HIFU system relates to the risk of burns, for example, when the internal ultrasound transducer is not coupled well to the arterial wall. To avoid such risk conventional intravascular HIFU catheters oftentimes employ an intravascular balloon, so as to ensure good coupling to the vessel wall. This, however, blocks the blood flow in the treated vessel during treatment, thereby limiting the duration of treatment and its effectiveness.

The technique of the present embodiments overcomes the above deficiencies by providing radiation-emitting system positioned away from a tissue target and optionally outside the body. For example, when the radiation is HIFU, it is not bound by a small contact area with the tissue because it is coupled to the skin, and can assure sufficient ultrasonic coupling, e.g., using impedance matching substances and the like. Thus, there is a wide range of possible frequencies and intensities that result in an effective modulation of the nerve. In various exemplary embodiments of the invention the method employs ultrasound at frequency of from about 400 kHz to about 4 MHz.

The technique of the present embodiments is advantageous also over other conventional techniques such as the aforementioned MRI or ultrasound image guided external HIFU, particularly in clinical situations in which the target tissue moves due to breathing, and/or when the required spatial accuracy is higher than providable by the ultrasound or MRI systems. A particular example is the case of renal nerve ablation for which the preferred spatial resolution is in the sub-millimeter range, which is not providable by speckle or spectral analysis. Such resolution is also not providable by standard MRI since the renal nerves move while the subject is breathing, and the long shutter time of standard MRI does not allow acquisition at sufficient spatial resolution of moving objects. While some modern MRI techniques allow the imaging of electron beam, these techniques are costly and technologically difficult to employ. Use of high enough power level to create detectable mechanical artifacts is also not desired, as indicated hereinabove.

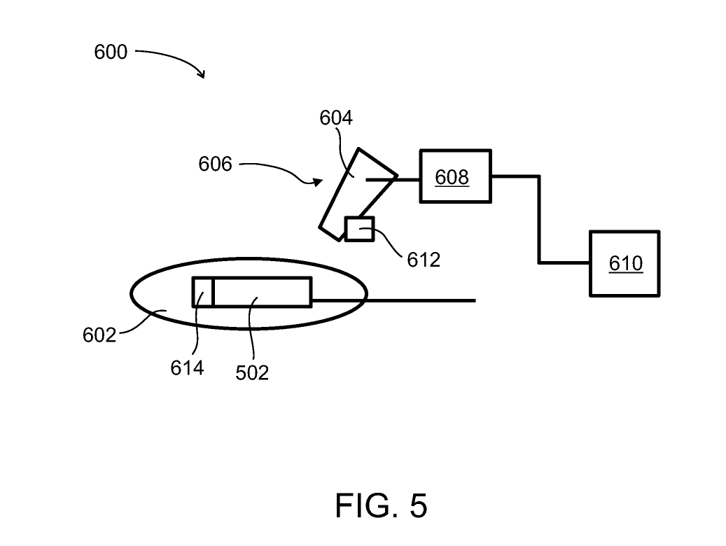

The embodiment of the invention in which scanning is performed along a predetermined path is also advantageous over conventional image guided techniques since conventional techniques typically require a manual control of the beam by the operator, and are therefore susceptible to human error. The employment of predetermined path according to some embodiments of the present invention overcomes this susceptibility since it may be performed automatically. Yet, use of semi-automatic means for controlling the scan along the path is not excluded from the scope of the present invention. For example, the system of the present embodiments may automatically track a movement of a renal artery, allowing the operator to shape the treatment path relative to the artery, without needing to deal with the movement of the artery due to respiration. In these embodiments the system optionally and preferably includes a computer screen in which an image of the artery is displayed in a static position, and the operator shapes the path around it. The system tracks the motion of the artery, and moves the treatment beam relative to the position of the artery according to the operator instructions, compensating automatically for organ movements. In some embodiments a controller (such as controller 604 in FIG. 5) directs an energy beam according to a combination of a) information provided by sensor-based aiming techniques as described with reference to a variety of embodiments presented herein, and b) operator provided information regarding positions of target tissues defined with respect to positions of the sensors. It is this combination that can enable an operator to designate a treatment path (designated relatively to positions of sensor(s) and/or position of a treatment target immobilized with respect to the sensors) on a graphic interface showing a static image, and have the system dynamically aim energy towards the target tissues designated by the operator on the static image, although the system, responding to target designations and/or other commands presented on the static image, is in fact engaging in real-time tracking of and aiming toward a moving target.

Since the tissue according to some embodiments of the present invention is fixated, the path may be set in advanced and be programmed into the scanning system, thus reducing risk of damaging tissues nearby the target tissue. Thus, instead of shaping the path of the treatment beam to the shape of the target tissue, the present embodiments take an opposite approach. The target tissue is shapes to a geometry in accordance with a preconfigured required shape. In some embodiments of the present invention the preconfigured shape of tissue is aligned with a preconfigured treatment path, or treatment points of the radiation beam. In some embodiments of the present invention, the path shape is defined parametrically, wherein during the procedure, shape parameters that are specific to the patient and organ are collected to determine the exact treatment locations.

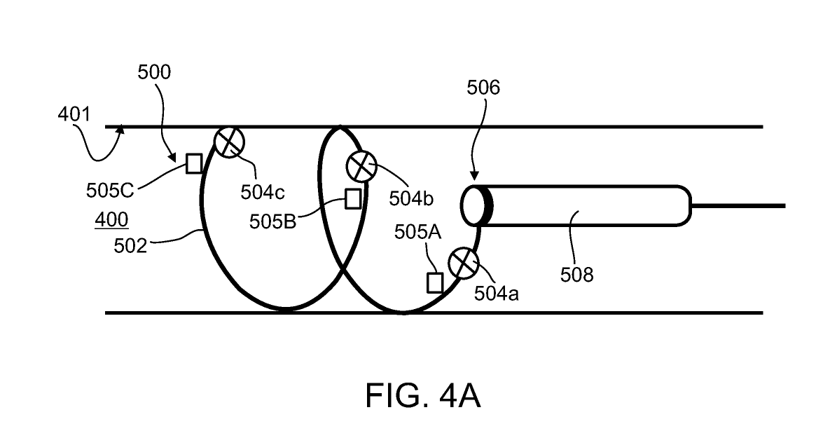

Reference is now made to FIG. 3, which is a flowchart diagram describing in greater detail a method according to some embodiments of the present invention, and to FIG. 4A, which is a schematic illustration of a catheter system, according to some embodiments of the present invention.

In FIG. 3, the method begins at 30 and continues to 11 at which the tissue is fixated as further detailed hereinabove. A representative example of the fixating procedure is illustrated in FIG. 4A which schematically illustrates a catheter system 500 having a shaped device 502 which may be mounted, for example, at a distal end 506 of a catheter 508, according to some embodiments of the present invention. In this representative example, shaped device 502 has a helical shape, which is particularly useful for fixating the internal wall 401 of a blood vessel such as, but not limited to, a renal artery. However, this need not necessarily be the case, since, for some applications, it may be desired to have a shaped device having a shape other than helical as indicated above.

The size of device 502 and catheter 508 is selected in accordance of the size of the artery to be treated. Optionally and preferably, multiple devices of different shapes and sizes are provided to allow the operator to select the most suitable device for the procedure.

The method optionally continues to 31 at which signals indicative of a relative position of the radiation-emitting system with respect to the shaped device are received. This may be achieved, for example, using one or more sensors 504a, 504b and 504c mounted on device 502 and/or catheter 508 and configured for sensing the position of the shaped device 502 within a living body (e.g., within a lumen 400 of a blood vessel) and for transmitting signals pertaining to this position. Optionally, sensors 504 may be energy sensors able to detect energy radiated by the radiation-emitting system. Optionally, the plurality of sensors 504 are not co-planar. It is noted that there is no need for a position sensor reporting an absolute position of the shaped device, because the system of the present embodiments is configured to direct the beam at, or at a known position with respect to, sensors 504. This embodiment is advantageous over conventional systems which do not employ fixation. Some embodiments employ a shaped device which is immobilized with respect to tissue by friction, pressure, or another method of attachment. Some embodiments employ a shaped device, such as for example an expandable shape device, which expands to assume a shape similar to that of an existing tissue, thereby optionally immobilizing one with respect to the other. (This may be contrasted to a use of sensors in prior art, where position sensors attached to a catheter tips allows determining the relative position of the tip with respect to the treatment system, but not with respect to the position of the target tissue itself.) This is because the offset between the tip and the target tissue is unknown, both in magnitude and in direction. Piezoelectric sensors, or PVDF sensors, or electro-optic sensors, or temperature sensors, or x-ray sensors, are a partial and exemplary list of types of sensors which might be used.

Optionally, the geometrical setting of the sensors 504 near a target site may be such that a target treatment path, relative to all or some of the sensors positions, is well defined. For example, in treating renal artery surrounded by nerves, sensors placed at inside the artery and touching the artery wall can have a predictable spatial relationship with positions of nerves whose denervation is desired. Therefore, using the sensors, a treatment path (e.g. the path of a denervating energy beam) can be defined around the artery by defining it with respect to sensor positions within the artery. In some embodiments sensors are placed inside an expanding cage in an artery, the sensors placed in a known relation to the cage known geometrical shape (for example two sensors in the axis of the cage, and one at a side touching the artery wall). Other clinical situations fit for such a system are the treatment of atrial fibrillation by pulmonary vein isolation, where sensors are placed at an expanding cage, at the distal end of an intravascular catheter, which is made to fit the entrance of the vein to the atrium, and the shape of the cage made to park at a known position relative to the entry point; sensors are positioned at the cage such that their position is fixed relative to the target tissue, or fixed relative to the shape of treatment (e.g. in the example above, at the arterial wall adjacent to the vein entrance)

The method optionally continues to 32 at which a non-damaging radiation is focused on the fixated tissue.

As used herein “non-damaging radiation” refers to radiation having intensity and duration selected such as not to cause irreversible modulation to the target tissue.

The use of non-damaging radiation is advantageous since it allows to adjust the scanning path and calibrate the radiation parameters (also called the “beam aiming parameters” herein) without causing damage or with minimal damage to non-targeted tissue.

In various exemplary embodiments of the invention the method continues to 13 at which the focused radiation, which some embodiments is non-damaging, is scanned along a predetermined path, as further detailed hereinabove. At 33 the radiation is sensed at or in proximity to the shaped device. This may be done using one or more radiation sensors, configured to respond to the radiation beam. Referring again to FIG. 4A, the present embodiments contemplate a configuration in which one or more of the sensors 504a, 504b and 504c are radiation sensors. In alternative embodiments, some or all of sensors 504 may be position sensors, such as, for example, sensors which detect a position-dependent electromagnetic field generated by a position-detection system.

Thus, in some embodiments a catheter system comprises one or more position sensor(s), optionally without any other type of sensor; in some embodiments a catheter system comprises one or more radiation sensor(s), optionally without any other type of sensor; and in some embodiments a catheter system comprises one or more position sensor(s) as well as one or more radiation sensor(s).

The radiation sensors may be positioned in a known geometrical relationship with the fixation structure of the shaped device. For example, for directing an external HIFU beam, a set of pressure sensors may be placed in known geometry with relation to the fixation structure of the shaped device. The radiation may initially scan the approximate target treatment area, and the beam parameters (e.g., phase shift, amplitude) as sensed by each sensor may be recorded. As a representative example, the beam parameters for which a sensor senses maximum pressure amplitude may be recorded, individually for each of the sensors. Thereafter, one or more such recordings may be correlated with the preplanned path of treatment and/or points of treatment.

In some embodiments of the present invention the position of the focal region relative to the sensor is calculated, for example, using a data processor, based on the signals received from the sensors. Optionally, such calculation is performed without calculating the absolute position of the sensors. From the geometrical relationship between the sensors and the fixation structure, and the information regarding the position of the focal region relative to the sensors, the method optionally and preferably calculates the position of the focal region relative to the fixation structure, hence also the position of the focal region relative to the fixated tissue.

Embodiments in which the absolute position of the sensors is also calculated are not excluded from the scope of the present invention. In these embodiments, the method may receive information pertaining to the location of the fixation structure with the body and uses this information, together with the geometrical relationship between the sensors and the fixation structure, for obtaining the location of the sensors.

In a representative example shown in FIG. 4A, device 502 is helical and comprises three or more radiation sensors, where one sensor (sensor 504a in FIG. 4A) is at the beginning of the helix, one sensor (sensor 504c in FIG. 4A) is at the end of the helix, and one sensor (sensor 504b in FIG. 4A) is approximately at the middle of the helix, optionally and preferably at a position that is not collinear with the other two sensors. Other arrangements and numbers of sensors are not excluded from the scope of the present invention.

The method may thus correct 34 the treatment path (e.g., location, radius) based on signals received from sensors 504a, 504b and 504c such that the treatment path or treatment points follow the shape of device 502 at predefined offset into the tissue. For example, the method may keep the focal region of the focused radiation at a distance of 0.5-3.5 mm outwardly from device 502 so as to assure treating the renal nerves lining the artery with reduced or no damage to the inner wall.

The sensors may communicate with the radiation-emitting system by wire or wireless communication. The present Inventors contemplate many types of sensors and sensor arrangements. In some embodiments, the sensors are arranged at a plurality of discrete locations relative to the shaped device, e.g., as illustrated in FIGS. 4A and 4B, and the sensing is therefore performed selectively at the location of the sensors.

In some embodiments, the radiation is sensed by imaging wherein a data processor executes an image analysis procedure so as to identify focal regions corresponding to the focused radiation. Preferably, the imaging is performed intracorporeally. For example, when the tissue is a blood vessel, a miniature intravascular imaging system is employed. The imaging system may be mounted for example, on the catheter. The imaging system may also be mounted on a trans-esophagus catheter or any other intracorporeal device.

Any type of imaging that can be used for detecting focal regions may be employed. When the radiation is HIFU radiation, the imaging preferably comprises ultrasound imaging, wherein the acquired ultrasound images are then processed to detect focal regions in the image. The focal region may be detected by identifying mechanical vibrations of the tissue in response to the focused radiation, by analyzing speckles in the image, by spectral analysis of the signal, or any other image analysis technique or combination of techniques.

In some embodiments of the present invention, the sensing is by reflecting the radiation outwardly and collecting the reflected radiation outside the body. In these embodiments, the shaped device may be mounted with one or more reflectors which reflect the radiation outwardly. For example, a reflector 505 (examples are labeled 505a, 505b and 505c in the FIG. 4A) may optionally be positioned at or near the location of one or more of sensors 504a, 504b and 504c. The reflectors may replace the sensors or they may be provided in addition to the sensors.

The reflected radiation may be sensed using a dedicated set of sensors arranged outside the body. Such sensors may be arranged, for example, on the radiation-emitting system. Alternatively or additionally, the radiation-emitting system, e.g., HIFU system, may be configured to receive the reflected radiation, e.g., by means of transceivers configured to receive radiation at the wavelength of the reflected radiation. The method may record the radiation parameters for each reflector, for example, when the corresponding reflected radiation is maximal. Use of reflectors is advantageous from the standpoints of cost and availability.

The present embodiments differ from diagnostic systems, such as diagnostic ultrasound, because it is not necessary to extract spatial resolution from the reflected radiation. Specifically, since the position of the reflector is known, only the radiation parameters (amplitude, phase, or other parameters) of the reflected radiation are analyzed. In some embodiments of the present invention the receiver has a narrow band which is adapted for the wavelength of the emitted radiation. This is unlike diagnostic systems, e.g., diagnostic ultrasound in which the bandwidth is made wide to improve signal to noise ratio.

In some embodiments of the present invention reflectors 505 are switchable and the method switches the reflectors on and off so as to associate the reflected radiation with each sensor. For example, a reflector may be made switchable by placing it in a capsule, e.g., within the structure of the catheter, and rotating it, e.g., mechanically or by applying a magnetic field, such that when it points to one direction it is considered in an “on” state and when it points to another direction it is considered in an “off” state.

In some embodiments of the present invention the reflector is encapsulated within a capsule which is fillable with fluid. These embodiments are particularly useful when the applied radiation is ultrasound radiation, whereby when the capsule is filled with liquid, the liquid vibrates with the ultrasound wave. Such an encapsulated sensor may be switch off by introducing gas into the capsule and switched on by introducing liquid into the capsule. Thus, for example, the capsules may be initially filled with liquid and the method may selectively introduce gas into the capsules to evacuate at least a portion of the liquid. Conversely, the capsules may be initially filled with gas and the method may selectively introduce liquid into the capsules evacuate at least a portion of the gas. Also contemplated, are embodiments in which the method repeats the procedure, namely introduce liquid after liquid evacuation and/or gas after gas evacuation. In any of these embodiments the method analyzes the resulting changes in the reflected radiation to associate the reflected radiation with individual capsules.

In some embodiments of the present invention the reflectors modulate the radiation upon reflection wherein different sensors may detect (and/or are selectively sensitive to) different modulations, and the method associates the reflected radiation with each sensor based on the modulation. Such modulation may include switching between sensor states at an identifiable frequency. For example, reflector 505a may be switched on and off periodically as a first rate, reflector 505b may be switched on and off periodically as a second rate, and reflector 505c may be switched on and off periodically as a third rate. The association of reflective radiation with a particular reflector may be achieved by filtering the reflected radiation according to the respective rate. Thus, the modulation encodes spatial information into the reflected radiation.

Once the scanning path is corrected, the method optionally and preferably loops back to 13, so that the operations 13, 33 and 34 are performed iteratively, until a predetermined accuracy level is achieved.

The method then proceeds to 35 at which the method receives calibration data, and to 36 at which the radiation is calibrated based on the received calibration data. The calibration data may be prepared in advance, for example, at laboratory calibration time or the like. A calibration system may move a radiation sensor in three-dimensions to cover all operational volume designed for the treatment. For each position of the sensor, the calibration system may focus the radiation beam onto the sensor, for example, by scanning the region in the neighborhood of the sensor until a desired reading, e.g., maximum amplitude, is obtained from the sensor. For each such position, the corresponding radiation parameters for that position are recorded. Representative examples of such parameters including, without limitation, phase shift of the radiation and intensity amplitude. The resulted data thus includes a set of position values (typically coordinates in three-dimensions) and corresponding radiation parameters. The data may be stored, for example, as a multidimensional matrix, prior to the execution of the medical procedure, or as factory settings.

In various exemplary embodiments of the invention the calibration 36 comprises correlating the calibration data with the radiation parameters at the relative position of the sensor with respect to the fixating structure or tissue. For example, the method may search for the expected relative position of each sensor based on the position values in the calibration data. This expected position typically deviates from the relative position of the sensor due to, e.g., body and environment distortions. The method then employs an interpolation algorithm for calculating the radiation parameters based on the relation between the expected positions from the calibration data and the known relative positions of the sensors.

Once the radiation is calibrated the method optionally and preferably loops back to 13, so that at least some of operations 13, 33, 34 and 36 are performed iteratively until a predetermined calibration accuracy is achieved. In other embodiments, the sensors read one or more transmitter elements, the system determines their parameters (such as, but not limited to, phase and amplitude), and iteratively loops over other elements, each time correlating the information to calibration information obtained in the laboratory, to analyze relative intensities, phases, and other such parameters of each portion of the whole transmitting system.

The method may then proceed to 37 at which the radiation is scanned along the treatment path so as to modulate the tissue. The fixation of tissue to the known geometry, the fixation of the sensors to the same geometry, and the communication between the radiation-emitting system and the sensors according to some embodiments of the present invention allows for an accurate treatment with reduced or no damage to non-target tissue. Optionally, the method may measure the parameters of only a portion of the transmitting elements, and determine the transmission parameters for treatment for all elements by means of algorithms such as interpolation or extrapolation of parameters.

The present Inventors recognize that during treatment, breathing, heartbeat, digestion, patient movement and other movements may alter the geometrical relationship between the beam transmitters and the fixated geometry of the target tissue.

In some embodiments of the present invention, the alignment between the fixated tissue and the treatment path is updated so as to compensate for tissue movement, or any other misalignment that may occur in time during treatment. In some embodiments, once a portion of the tissue is modulated, the method loops back from 37 to 32, the radiation is reduced to non-damaging levels and the method corrects the path based on the sensed radiation and/or calibration data as further detailed hereinabove. The rate and duration at which the path is updated is optionally and preferably predetermined. For example, the method may operate at a period P and duty cycle D, wherein during the period P, the method scan to modulate the tissue for a time-interval P·D, and performs path updates for a time-interval P·(1−D). Typically, but not necessarily, P is from about 10 milliseconds to about 1 second, and D is from about 0.5 to about 0.95. In some embodiments, alignment between fixated tissue and the treatment path is updated whenever movement of the tissue exceeds a predetermined portion of the size of the region of focus of the beam. The duration in which the target tissue movement is bigger than that of a predetermined portion of the focal size may be preconfigured or measured by analyzing the results at 13.

A representative example of the operations during a period P is as follows. The method initially determines the location of the sensors, treatment path and radiation parameters as further detailed hereinabove. The method stores the positions of the sensors and modulates the tissue for the tissue-modulation time-interval P·D. The radiation is then reduced to a non-damaging level for the path-update interval P·(1−D). During the path-update time-interval, the method focuses the radiation onto one of the previously stored locations of the sensors. For each such location, the method moves the focal region at the vicinity of the location, preferably in three-dimensions (e.g., at each of the six directions: front, back, left, right, up and down), to allow the sensor to responsively sense the change in radiation. The method may use interpolation or extrapolation methods for calculating the parameters of transmitters that were not measured during the measurement cycle. The method may use interpolation or extrapolation algorithms to update transmission parameters so as to adopt preferable treatment positions with respect to target tissue movement in between measurement cycles.

For example, when the radiation in HIFU, the sensor may be a pressure sensor which senses changes in the pressure amplitude when the HIFU focal region moves at the vicinity of the sensor. Once the sensed amplitude reaches a local maximum, the method determines that the HIFU is directed onto the sensor and stores the updated location or direction, and optionally also other radiation parameters. The updated information is then used for updating the treatment path. It is to be understood that this procedure may also be used for other sensed parameters and other types of radiation as indicated above.

The radiation level is then increased back to the damaging level and a new cycle is executed wherein the tissue is modulated along the updated path. The above procedure is optionally and preferably executed for each of the sensors. In some embodiments of the present invention locations of two or more (e.g., all) the sensors are updated during a single path-update time-interval, and in some embodiments of the present invention the locations are updated intermittently, namely the locations of at least two sensors are updated during different cycles.

In some embodiments of the present invention, signals received during consecutive measurements, either in the same cycle or consecutive cycles, are analyzed so as to determine the motion vector and optionally also the acceleration of the focus relative to the shaped device. The motion vector may then be used to calculate, typically by a data processor, the path of focal region, to compare the calculated path with the treatment path, and to update the scan based on the comparison. The scanning is optionally performed continuously along the preconfigured treatment path. Performing the scan in a continuous manner is advantageous, because it reduces the probability of damage to non-target tissue without decreasing the effectiveness of the treatment. In some embodiments, estimated path parameters such as position, velocity, and acceleration of each sensor are calculated, and during this calculation, the system updates the position of the treatment beam according to previously calculated parameters

Also contemplated, are embodiments in which the radiation is sensed also during the tissue-modulation time-interval. The method may update the path or intensity or any other transmission parameter during treatment, namely without reducing the radiation level of each of the sensors during treatment, or, more preferably, the method may reduce the radiation once the method determines that the difference between the sensed radiation and the expected radiation, for a particular sensor, is above a predetermined thresholds. Thus, in these embodiments, the method loops back from 37 to 33 at which the radiation is sensed. If the difference between the sensed and expected radiation is below the threshold, the method returns directly to 37. If the difference between the sensed and expected radiation is not below the threshold, the method loops back to 32, the radiation is reduced to a non-damaging level and a cycle or period P is initiated as further detailed hereinabove. For clarity of presentation, transitions corresponding to these embodiment (e.g., from 37 to 33, and from 33 to 32) are not shown in FIG. 4A, but one of ordinary skills in the art, provided with the details described herein would know how to adjust the flowchart to show such transitions.

The method ends at 40.

Reference is now made to FIG. 4B, which is a schematic illustration of a shaped device according to some embodiments of the present invention. Shaped device 502 is optionally an expandable shaped device 512, as shown in the figure. The embodiment shown in FIG. 4B is designed to adopt a contracted profile during insertion, for example by being constrained to a narrow profile by being contained in a guiding sheath (not shown), and to adopt an expanded profile (as shown in the figure) when the expandable portion of the device is extended beyond a distal end of the constraining guiding sheath, and/or when the guiding sheath is retracted from around the expandable portion. As mentioned above, similar devices may be made to assume an expanded configuration in response to mechanical, thermal and/or electrical activation. In an optional method of use, expandable shaped device 512 is inserted into a body lumen, such as for example a blood vessel or an esophagus, and there caused to expand. The expanded device 512 may be expanded until its expandable arms 519 come in contact with, and optionally slightly stretch, walls of the lumen in which it has been inserted. Several purposes may be accomplished. One is to immobilize device 512 within the lumen into which it is inserted, so that sensors carried by device 512 come to have a fixed position with respect to the lumen and whatever is connected to walls of the lumen. Another is to enable manipulation of the position of the lumen walls and whatever is connected to them by manipulating the position of device 512, for example to move an organ into which device 512 out of danger while a nearby organ is being treated. Yet another is to cause the lumen walls to assume a pre-planned shape, for example to facilitate treatment of the walls and/or associated tissues. Yet another is to calculate target tissue position relative to sensors mounted on the device, positioned in a shape known to correspond to a shape of the tissues.

It is to be understood that the specific designs shown in FIGS. 4A and 4B are exemplary only, and not to be considered limiting. In particular with respect to FIG. 4B, it is noted that any expandable cage-like and/or mesh-like expandable structure may be used, for example, to fix a surrounding blood vessel to a pre-determined shape, for example to a shape which is cylindrical, or to a shape which is squared with rounded corners, and which is optionally approximately co-axial to the catheter.

Optionally, expandable shaped device 512 may comprise one or more sensors 504 and/or reflectors 505, as discussed hereinabove and as shown in FIG. 4B.

Reference is now made to FIG. 5 which is a schematic illustration of a system 600 for modulating tissue of an internal organ in vivo. System 600 comprises one or more shaped devices adapted for being introduced into a living body 602 and being configured for fixating a tissue thereon so as to shape the tissue generally according to a shape of the device. One or more of the shaped devices may be for example, device 502, in which case system 600 preferably comprises catheter system 500 including shaped device 502, catheter 508 and optionally also sensors 504a-c and/or reflectors 505a-c, as further detailed hereinabove.

While FIG. 5 shows a system including a single shaped device, it is not intended to limit the scope of the present invention to such configuration. For example, the system may comprise a plurality of shaped devices, whereby different devices are adapted for being introduced to different locations in the body. For example, different devices may be adapted for being introduced into different blood vessels. In some embodiments of the present invention different devices are adapted for being introduced into different locations in the same blood vessel.

System 600 further comprises a radiation-emitting system 604 configured for emitting radiation from a location 606 external to body 602 and focusing the radiation on the fixated tissue. In various exemplary embodiments of the invention system 600 comprises a scanning system 608 operative to scan the radiation over the fixated tissue. Scanning system 608 may move radiation-emitting system 604 and/or it may divert the radiation using an arrangement of redirecting elements (not shown), such as, but not limited to, mirrors, prisms, diffractive elements and the like. In various exemplary embodiments of the invention the scanning is performed automatically, e.g., using a controller 610 configured for controlling radiation-emitting system 604 and scanning system 608 such that the scan is along a predetermined path corresponding to the shape of device 502. Controller 610 may include or be supplemented with a data processor configured for performing the calculations described herein, and scanning system 608 may optionally be part of controller 610.

It is noted that in some embodiments the functionality of scanning system 608 is included within functions of controller 610, while in other embodiments (where scanning system 608 is a mechanical device, for example), beam aiming operations may require participation of emitter 604 and also of scanning device 608, both under control of controller 610. For simplicity of exposition, in this document references to beam controlling operations effected by controller 610 should be understood as including, in some embodiments, operations in which scanning device 608, directed by controller 610, participates as well.

When the system comprises a plurality of shaped devices scanning system 608 scans the tissues fixated on at least some of each of the shaped devices, more preferably on each of the shaped devices. Optionally, each such tissue is treated separately, so as to reduce the risk of damaging neighboring tissue. Alternatively, a path may be selected to allow treating two or more tissues that are fixated on different devices during the same scan. For example, the path may have the shape of a figure of 8 around and between two blood vessels (e.g., an artery and a vein). Optionally, such path is preplanned. Optionally, the path is automatically selected based on the geometry of the fixated tissues and shaped devices. Optionally, the path is manually chosen by an operator, and the system uses the sensor device for omitting selected organs (e.g., vital organs such as, but not limited to, artery, vein, urethra) that are not to be affected, and/or for compensating for organ movement.

When device 502 comprises one or more sensors (e.g. sensors 504, shown in FIG. 4A and FIG. 4B) for sensing the radiation or position of the shaped device, controller 610 receives signals from the sensor(s) and controls radiation-emitting system 604 and scanning system 608, responsively to the received signals, as discussed in further detail hereinbelow.

When device 502 comprises one or more reflectors 505 for reflecting the emitted radiation, system 600 may comprise one or more radiation sensors or receivers 612 configured for sensing the reflected radiation at one or more sensing locations external to the body, as further detailed hereinabove. In these embodiments, controller 610 receives the signals from the radiation sensor 612 or receiver and controls radiation-emitting system 604 and scanning system 608, responsively to the received signals.

In various exemplary embodiments of the invention controller 610 is configured for calibrating the radiation. In some embodiments of the present invention the calibration is performed in response to the signals received from the sensors. In some embodiments of the present invention controller 610 accesses prerecorded calibration data having a plurality of entries, each entry comprising a set of radiation parameters associated with a three-dimensional coordinate. Controller 610 searches the data for a 3D coordinate corresponding to a sensing location and extracts a respective set of radiation parameters. Controller 610 then calibrates system 604 based on the respective set of radiation parameters.

In some embodiments, system 600 comprising an intracorporeal imaging system 614 configured for imaging the fixated tissue and regions in proximity thereto. In these embodiments, controller 610 analyzes imagery data received from intracorporeal imaging system 614, and identifies focal regions corresponding to the focused radiation, as further detailed hereinabove. In an exemplary embodiment shown in FIG. 5, intracorporeal imaging system 614 is shown as being contiguous to device 502, yet this configuration is exemplary and not limiting. Imaging system 614 might, for example, be positioned in a first organ and imaging system 614 in a nearby second organ.

The present embodiments also contemplate configurations in which the treatment system 604 is intracorporeal, and configurations in which both intracorporeal and an extracorporeal systems are employed. In some embodiments, device 502 aligns a treatment tool, such as an electrode, an intravascular HIFU reflector or transducer, or any other mechanism that causes treatment, to move along the predetermined path within the body 602. For example, device 502 may include an RF electrode movable along the catheter 508. Alternatively, a set of treatment tools, such as RF electrodes are fixed on the length of the fixation structure such that each affects a location along the shape of device 502. Also contemplated are embodiments in which an intravascular HIFU system (not shown) with a beam of substantially less than 360°, is mounted on device 502 or catheter 508. The intravascular HIFU system may scan the radiation along the predetermined path, e.g., by a reflector or by moving the transducer, so that the focal region moves along the path or along the shape of device 502.

In some embodiments of the present invention multiple devices are positioned in organs in the vicinity of target tissue for enabling treatment path of a treatment beam to be conducted safely and without harming non-target tissue. For example, in some embodiments for treating atrial fibrillation, a system includes one or more catheters inserted into the initial portion of a pulmonary vein, and a trans-esophageal device inserted into an esophagus. During treatment, the system computes the path of the treatment beam in a way that will not excessively heat the esophagus of the patient. The system may additionally alert the operator for danger of over-heating the esophagus if it identifies that treatment path will hit the esophagus, and may suggest altering the geometrical configuration of the esophagus by moving it, or by altering the patient position. Other examples include a system of multiple catheters for renal denervation, one inserted into the artery, and the other into the vein; once treatment is operated, the system processor assures the path of beam is optimal in energy levels to not harm vein or artery, while conducting a path similar for example to the figure “8”.

In some embodiments of the invention, a system comprises multiple energy transmitters. During treatment, the system conducts repeated cycles of measurement and treatment as described above. In some clinical situations, some of the transmitters' beams are obstructed from reaching target tissue during a portion of the treatment, or during all of it. The system uses sensor readings made during the measurement cycles to determine whether transmissions from each transmitting element can reach the target, and temporarily shuts down the ineffective elements during associated treatment cycles. In this way the system processor can accurately determine the energy dose being delivered to the target tissues, and can calibrate energy levels of transmissions and duration of transmissions accordingly. Additionally, in this way obstructing organs are not affected by unwanted beam portions that might hit and damage them.

In some embodiments of the present invention the energy projector comprises a plurality of transmitting units. Examples are a phased array or annular array elements of a HIFU transmitter. The ultrasound energy which the transmitters emit travels in the speed of sound in the tissue, and therefore each element transmission takes time to reach the sensors. The system in this embodiment optionally measures each element's transmission concurrently at all sensors to optimize measurement cycle duration. In some embodiments, the system iterates the transmissions in a manner that allows transmitting from consecutive elements without waiting for the previous element transmission to reach the sensors; this can be achieved by choosing the next element to transmit to be close by to the previous one, thereby ensuring the sensing order of the beams will be similar to the transmission order.

In some embodiments of the present invention, the system uses measurements of delay between transmissions to multiple sensors with relatively known geometric relationship on the device. The system then calculates required delay of transmission to target tissue, optionally without determining the location of sensor, or target tissue. This method takes into account beam aberations that may behave differently from one transmitting element to another. By measuring the delay from multiple sensors, the system calculates the required delay for a nearby target position, for each transmitting element—thereby synchronizing all elements to hit target in concert. This method is advantageous, as it does not require imaging of target or beam, and does not require any measurement of location of sensors, target tissue or beam, yet enables conducting accurate beam treatment.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of modulating tissue of an internal organ in vivo, comprising:
inserting an expandable shaped device having at least one arm into an intrabody lumen of a patient;
expanding said expandable shaped device to attach said at least one arm to a wall of said intrabody lumen, said wall being attached to a target tissue;
using at least one sensor on said at least one arm to measure a motion of said wall;
calculating a motion of said at least one arm; and
adjusting a radiation transmitted from high intensity focused ultrasound (HIFU) source to said target tissue so as to modulate the target tissue according to said calculated motion;
wherein said HIFU source is an external to the body of said patient.

2. The method of claim 1, wherein expanding said tissue on said shaped device comprises shaping the tissue generally according to a shape of said expandable shaped device.

3. The method of claim 1, wherein expanding said tissue on said shaped device comprises causing said device to conform to a pre-known size and shape of said tissue.

4. The method of claim 1, wherein expanding said tissue on said shaped device comprises immobilizing said tissue with respect to said expandable shaped device.

5-8. (canceled)

9. The method according to claim 1 further comprising scanning said focused radiation along a predetermined path corresponding to a shape of said expandable shaped device in said intrabody lumen so as to form a modulation pattern on the tissue.

10. The method according to claim 9, wherein said scanning comprises moving said HIFU source.

11. The method according to claim 9, wherein said HIFU source is a phased array radiation-emitting system.

12. The method according to claim 9, further comprising receiving signals indicative of a relative position of said HIFU source with respect to said expandable shaped device, wherein said scanning is responsively to said relative position.

13. The method according to claim 9, further comprising sensing said radiation at or in proximity to said expandable shaped device, and correcting said path responsively to said sensing.

14. The method according to claim 13, wherein said expandable shaped device comprises a sensor operable to detect and report energy transmitted by said HIFU source.

15. (canceled)

16. The method according to claim 1, wherein said expandable shaped device comprises a portion sized and shaped to deploy as a helix biased against an inner wall of a blood vessel.

17-20. (canceled)

21. The method according to claim 13, wherein said sensing is performed selectively at a plurality of discrete locations.

22. The method according to claim 13, wherein said sensing comprises reflecting said radiation outwardly and collecting said reflected radiation at a receptor distant from said reflectors.

23. The method according to claim 22, further comprising modulating a waveform of said radiation so as to encode spatial information therein; wherein said modulating of said reflected radiation comprises periodically modifying reflectivity of a reflector with respect to energy arriving from a particular direction; further comprising modifying reflectivity of a plurality of reflectors with differing periodicity.

24-25. (canceled)

26. The method according to claim 13, wherein said sensing comprises imaging and the method further comprises operating a data processor to execute an image analysis procedure so as to identify focal regions corresponding to said focused radiation.

27-29. (canceled)

30. The method according to claim 13, further comprising, prior to said modulation of the tissue, operating said HIFU source to emit non-damaging radiation, wherein said correction of said path is performed during said emission of said non-damaging radiation.

31. (canceled)

32. The method according to claim 30, further comprising repeating said emission of said non-damaging radiation and said modulation intermittently.

33. (canceled)

34. The method according to claim 9, wherein said predetermined path forms a non closed loop spanning over 360 degrees about a longitudinal axis.

35. (canceled)

36. The method according to claim 1, wherein the tissue is a neural tissue and said modulation comprises denervation.

37. The method according to claim 36, wherein said nerve is part of an autonomic nervous system.

38. The method according to claim 36, wherein said nerve is selected from the group consisting of a nerve leading to a kidney, a sympathetic nerve connected to a kidney, an afferent nerve connected to a kidney, an efferent nerve connected to a kidney, a renal nerve, a renal sympathetic nerve at a renal pedicle, a nerve trunk adjacent to a vertebra, a ganglion adjacent to a vertebra, a dorsal root nerve, an adrenal gland, a motor nerve, a nerve next to a kidney, a nerve behind an eye, a celiac plexus, a nerve within a vertebral column, a nerve around a vertebral column, nerve extending to a facet joint and a celiac ganglion.

39-43. (canceled)

44. A system for modulating tissue of an internal organ in vivo, comprising:
a expandable shaped device adapted for being introduced into a living body and being configured for fixating a tissue thereon so as to shape the tissue generally according to a shape of said device;
a HIFU source configured for emitting radiation from a location external to the body and focusing said radiation on the fixated tissue;
a scanning system operative to scan said radiation over said fixated tissue; and
a controller, configured for controlling said HIFU source and said scanning system such that said scan is along a predetermined path corresponding to said shape of said device so as to from a modulation pattern on the tissue; and
at least one sensor mounted on said expandable shaped device and configured for sensing at least one of: a position of said expandable shaped device within a living body, and radiation emitted by said HIFU source, wherein said controller is configured for receiving signals from said at least one sensor and for controlling said HIFU source and said scanning system, responsively to said signals;
wherein said expandable shaped device comprises an expandable portion sized and shaped to bring elements of said expandable portion into contact with an inner wall of a blood vessel when said expandable portion is expanded within said blood vessel.

45. (canceled)

46. The system according to claim 44, wherein said at least one sensor comprises a plurality of sensors arranged at a plurality of discrete locations over said expandable shaped device.

47. The system according to claim 44, further comprising:
at least one reflector mounted on said expandable shaped device and configured for reflecting radiation emitted by said HIFU source; and
at least one radiation sensor configured for sensing said reflected radiation at one or more sensing locations external to said expandable shaped device;
wherein said controller is configured for receiving signals from said at least one radiation sensor and for controlling said HIFU source and said scanning system, responsively to said signals;
wherein said at least one radiation sensor comprises a sensor adapted to be located outside said body.

48-50. (canceled)

51. The system according to claim 44, wherein said controller is configured for calibrating said radiation responsively to said sensing; wherein said controller is configured to access prerecorded calibration data having a plurality of entries, each entry comprising a set of radiation parameters associated with a three-dimensional coordinate, and to search said data for a three-dimensional coordinate corresponding to a sensing location so as to extract a respective set of radiation parameters, wherein said calibrating is also based on said respective set of radiation parameters.

52-53. (canceled)

54. The system according to claim 44, further comprising: an intracorporeal imaging system configured for imaging said fixated tissue and regions in proximity thereto, wherein said controller is configured for analyzing imagery data received from said intracorporeal imaging system, and identifying focal regions corresponding to said focused radiation.

55-59. (canceled)

60. A system for modulating tissue of an internal organ in vivo, comprising:
a expandable shaped device adapted for being introduced into a living body;
a HIFU source configured for emitting radiation from a location distant from said expandable shaped device and for focusing said radiation on said expandable shaped device;
at least one sensor mounted on said device, and being configured for sensing said radiation; and
a data processor, configured for analyzing signals received from said at least one sensor and calculate at least one of: a relative location, a set of phases for phase array transmission for a focal region of said radiation, and a distance of a focal region of said radiation.

61. The system of claim 60, wherein said HIFU source configured for scanning the tissue and wherein said data processor is also configured for calculating a scanning path of said focal region.

62. (canceled)
